(19) United States
(12) Patent Application Publication
(10) Pub. No.: US 2019/0069921 A1
(43) Pub. Date: Mar. 7, 2019

(54) Method and Apparatus for Venous Blood Clot Disruption

(71) Applicant: EndoVention, Inc., San Francisco, CA (US)

(72) Inventors: Shaun L. W. Samuels, San Francisco, CA (US); Peter Yorke, San Francisco, CA (US)

(21) Appl. No.: 16/072,007

(22) PCT Filed: May 5, 2017
(86) PCT No.: PCT/US17/31205

§ 371 Date: Jul. 23, 2018

Related U.S. Application Data
(60) Provisional application No. 62/332,540, filed on May 6, 2016.

(51) Int. Cl. A61B 17/3207

(52) U.S. Cl. CPC A61B 17/320725; A61B 2017/2215

(57) ABSTRACT

A device and method for clot disruption in deep venous thrombosis is disclosed herein. The present technology is directed toward a device for clot maceration that includes a longitudinal member, in the form of a catheter, containing radially arrayed elements which, when positioned within a clot, are moved longitudinally, bidirectionally, and rotated in order to disrupt and macerate the surrounding clot. This allows the fragmented clot to be easily removed through a separate device; alternatively this renders the clot significantly more amenable to treatment with fibrinolytic agents given the markedly enhanced surface area of the fragmented clot. The radially arrayed elements may have a variety of forms, with a preferred embodiment containing bristles shaped specifically to engage vein valves in such a way as to cause no damage to them, while simultaneously prompting the clearance of clot from the valve sinuses.

### TECHNICAL FIELD

The current invention is directed to medical devices, and, more specifically novel devices for disrupting clots in the vascular system.

### BACKGROUND OF THE INVENTION

On occasion, blood clots may form within the vascular system. When this occurs in the venous system, the disorder is referred to as Deep Venous Thrombosis, or DVT. DVT represents a significant medical problem, occurring in over a million people each year.

The standard treatment for DVT is anticoagulation. Anticoagulation alone, however, is not very effective in resolving the blood clots that are already present, and instead prevents the propagation of blood clot and hence greatly reduces the risk that blood clot will break off and travel to the lung, a dangerous condition known as pulmonary embolus. The disadvantage of this form of treatment, however, is that the clot that has already formed may remain in place indefinitely, prolonging the pain and swelling usually associated with DVT. Furthermore, the unresolved clot eventually scars the vein, leading to chronic problems of pain, swelling, and ulceration, known as post thrombotic syndrome.

In response to these limitations of the current treatment, procedures have been devised to directly address the blood clots and facilitate their removal or dissolution. To that end, catheter directed thrombolysis, in which a catheter is advanced through the area of blood clot and a fibrinolytic agent, such as Tissue Plasminogen Activator, or TPA, is infused. This slowly dissolves the blood clot, typically over a period of one to two days. While this technique is often highly effective, it has many limitations. First, it is a relatively slow process, requiring very careful inpatient monitoring for the duration of the procedure, and blood monitoring at frequent intervals, both of which are associated with high cost and significant patient discomfort. Furthermore, there is significant risk associated with the infusion of fibrinolytic agents, as they can have systemic effects that may lead to serious, even fatal, bleeding.

An alternative to catheter-directed thrombolysis, which also directly addresses the blood clots, is percutaneous mechanical thrombectomy. In this technique, a device is introduced through a small skin incision into the vein and the device is advanced through the lumen of the vein, traversing the area occupied by clot. The device is actuated and the portion of the catheter which traverses the clot, said portion assuming a configuration designed for the purpose, breaks up the clot into smaller pieces to be removed. The limitation of these devices, however, is that their design does not take into account the unique structure of the veins, which includes the presence of vein valves. Addressing blood clot within the region of the vein valves requires a specialized method of engagement by a mechanical thrombectomy device, and this does not exist among the devices currently available.

It would therefore be desirable to have a clot disruption device specifically designed to remove clots from veins, shaped and configured to engage vein valves in such a way as to cause no damage to the valve and surrounding tissue. Accordingly, there is a need in the art for such a clot disruptor, as one does not presently exist in the art.

### SUMMARY OF THE INVENTION

The present invention provides a device directed toward treatment of deep venous thrombosis. The device consists of a longitudinal component, in the form of a catheter, with a generally hollow central lumen and specialized bristles or filaments radially arrayed circumferentially around the catheter body. In each preferred embodiment, these bristles or filaments have shapes specialized for the engagement of vein valves, such that movement of the device in any direction macerates the clot typically trapped at the level of the valve sinus, and simultaneously displaces the clot toward the lumen of the vein.

Such a device is not described in the prior art. Such clot disruption, without simultaneous clot extraction or aspiration, would be performed in an occlusive environment, either through use of a second occlusive device, or within an occluded segment of vein whereby the fragmented clot would be unable to embolize to the lungs.

These and other features, aspects, and advantages of the present invention will become better understood with reference to the following detailed description when considered in association with the accompanying drawings and claims.

### BRIEF DESCRIPTION OF THE DRAWINGS

FIG. 1 shows a venous clot disruptor device.

FIG. 2 shows a cross-sectional end view of a venous clot disruptor device.

FIG. 3 shows a venous clot disruptor device.

FIG. 4A shows a venous clot disruptor device in a collapsed state within a vessel.

FIG. 4B shows a venous clot disruptor device of FIG. 4A in an expanded state within a vessel.

FIG. 5 shows a venous clot disruptor device in an expanded state including additional groups of macerating elements within a vessel.

### DETAILED DESCRIPTION OF THE INVENTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Bristles of the inventive clot disruption device are generally soft and flexible extending outward from the catheter shaft. The bristles are resilient and sized to permit compression and passage of the disruption device out of, and back into, the distal end of an introducer lumen. Further, the bristles are configured to permit disruption of the venous clot while avoiding or minimizing damage to the vessel wall and vein structures. The bristles are configured to engage a venous clot located at a venous valve.

Bristles may be any suitable shape to dislodge and macerate a clot including straight, curved, or bent. In a preferred embodiment, the bristles are curved so that the tip of each bristle is not perpendicular to the vessel wall and does not contact the vessel wall while disrupting the clot; preferably the bristles are curved so that the tip of each bristle is oriented parallel to the vessel wall; preferably the bristles are curved so that the tip of each bristle is oriented away from the vessel wall; preferably the bristles are curved so that the tip of each bristle is oriented back towards the catheter shaft. In another preferred embodiment the bristles are straight so that the tip of each bristle is directed towards the vessel wall; in one embodiment the straight bristles are firm to engage and macerate a clot; in one embodiment the straight bristles are soft and flex to direct the bristle tip away from the vessel wall upon contact with the vessel wall. In another embodiment the bristles are bent or angled permitting the tip of each bristle to be aimed in any direction including directly back towards the catheter shaft; in one embodiment the bristles are bent at a 90 degree angle; in one embodiment the bristles are bent at a 45 degree angle; in one embodiment the bristles are bent at a 135 degree angle.

In yet another embodiment the bristles double-back to the catheter shaft so that the bristle is coupled to the catheter shaft at two points to form a loop. The bristle ends may be coupled to the catheter shaft at the same point or different points along the catheter shaft; in one embodiment the bristle ends are coupled at the same origin point on the catheter shaft to form a tear-shaped loop; in one embodiment the bristle ends are coupled at separate origin points on the catheter shaft to form a “D”-shaped loop with the catheter shaft. The ends of each bristle may be coupled to the catheter shaft so that the central axis of the loop is at any suitable angle with respect to the longitudinal direction of the catheter; in a preferred embodiment the axis of the bristle loop is perpendicular to the longitudinal direction of the catheter; in a preferred embodiment the axis of the bristle loop is parallel to the longitudinal direction of the catheter.

Bristles may be angled outward from the catheter shaft at any suitable angle from between nearly zero degrees (directed up and away from the catheter tip) to nearly 180 degrees (directed down towards the catheter tip). Preferred angles of engagement between the bristles and catheter shaft are 90 degrees (perpendicular to the shaft), 45 degrees, and 135 degrees. The angle formed between the bristle and catheter shaft may be selected based on the shape and orientation of the bristle. For example, to reach a point located at 135 degrees from the catheter shaft, straight bristles may be coupled to the catheter shaft at 135 degrees or alternatively bent bristles may be coupled to the catheter shaft at 90 degrees to accommodate a bristle bend of 45 degrees.

Bristles may be of any suitable length and number to disrupt a clot. Preferably bristles are sized to ensure disruption of the clot at all portions of the lumen from the centerline to the vessel wall. In one embodiment the bristles are substantially all the same length; in one embodiment bristles are various lengths. A cluster of bristles may be configured at one or more points along the catheter shaft to assist with clot dislodgement and maceration. Clusters may be positioned at one or more location radially around the catheter shaft or at one or more location longitudinally along the shaft. It is envisioned that more than one type of bristle or more than one type of bristle cluster may be used on a single catheter shaft. Spacing between venous valves may be between about 1 centimeter and about 16 centimeters. Clusters of bristles may be spaced at any appropriate position on the catheter shaft to accommodate one valve or more than one valve; preferably clusters of bristles may be spaced about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, or about 16 cm apart. In this fashion, the different valves and intervening valve segments may have a clot or more than one clot disrupted separately. If the bristles to be distributed over longer lengths, then their respective interactions with adjacent vein valves simultaneously might not be optimal, as one set of bristles engages a valve appropriately, the adjacent set of bristles may not be aligned correctly.

Bristles may be formed of any suitably flexible and resilient material. Bristles are preferably formed of polymeric material or metallic material. Bristles may be comprised of polymers such as nylon, polyimide, polyethylene, polyethylene terephthalate, polyurethane, polypropylene, expanded polytetrafluoroethylene, and polyether ether ketone. Bristles may be formed of metals and metal alloys such as titanium, stainless steel, and nickel titanium. In one preferred embodiment the bristles are made of nylon; in one preferred embodiment the bristles are made of a nickel and titanium (nitinol) shape memory alloy. Bristle flexibility and resilience may vary along the length of the bristle by varying for example, the thickness or other characteristics of the bristle. Bristles may have varying degrees of firmness or softness and each bristle may have a gradient of flexibility along its length, for example firm at the point of attachment to the catheter shaft but soft at the tip to minimize damage to the vessel wall.

Each bristle may be substantially linear or may have one or more protrusions along the length of the bristle to enhance clot disruption and maceration. Additionally, the tip of each bristle may be solid or may be frayed, split, splintered, or fractured into multiple tips to minimize damage to the vessel while enhancing clot maceration.

In an alternative embodiment, two ends of a flexible and resilient filament are attached to the catheter at two separate points that are movable in relation to one another so that the distance between the filament ends may be shortened or extended, resulting in the filaments elongating and coming to rest along the catheter shaft in the extended configuration, or the filaments flared to their unconstrained nominal configuration when the catheter shaft elements are slidably foreshortened. By shortening the distance between the filament ends, the filament takes on an unconstrained shape resulting in a filament blade suitable for dislodging and macerating a venous clot. In this embodiment the first end of the filament is coupled to a point on the catheter near the catheter tip and the second end of the filament is coupled to a slidable hub located proximally away from the catheter tip.

In this embodiment the catheter is introduced into the lumen of the vein with the filament ends apart from each other longitudinally to allow the filament to have a low profile, oriented along the catheter axis. Once positioned at the venous clot, the slidable hub is advanced moving the second end of the filament closer to the first end and permitting the filament to assume its unconstrained shape so the filament blade can engage the vein valve.

Clot disruption devices having filament blades may have any suitable number of filaments; in a preferred embodiment the clot disruption device has between one and thirty filaments; the clot disruption device may have 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 filaments. In a preferred embodiment the clot disruption device has a single filament; in another preferred embodiment the clot disruption device has two filaments. Preferably the clot disruption device has three or more filaments; preferably the clot disruption device has four or more filaments; preferably the clot disruption has six or more filaments. The clot disruption device may have an odd number or an even number of filaments; in a preferred embodiment the clot disruption device has an even number of filaments; in another preferred embodiment the clot disruption device has an odd number of filaments. In one preferred embodiment the clot disruption device has three filaments; in one preferred embodiment the clot disruption device has four filaments; in one preferred embodiment the clot disruption device has six filaments. In embodiments with more than one filament, preferably the filaments are equally spaced around the catheter shaft.

Filament blades of the clot disruptor device may be present alone or in combination with additional filaments or elements to enhance clot disruption and maceration. These additional filaments or elements may be made of polymeric or metallic material and may be the same or different material compared with the filament material. In one embodiment, the filament blade comprises concentrically-oriented elements; in another embodiment the filament blade comprises a lattice-work of elements; in another embodiment the filament blade comprises a web of elements; in another embodiment the filament blade comprises one or more transverse elements. In one embodiment the filament is made of a shape-memory metal and comprises interconnected elements comprised of a polymeric material.

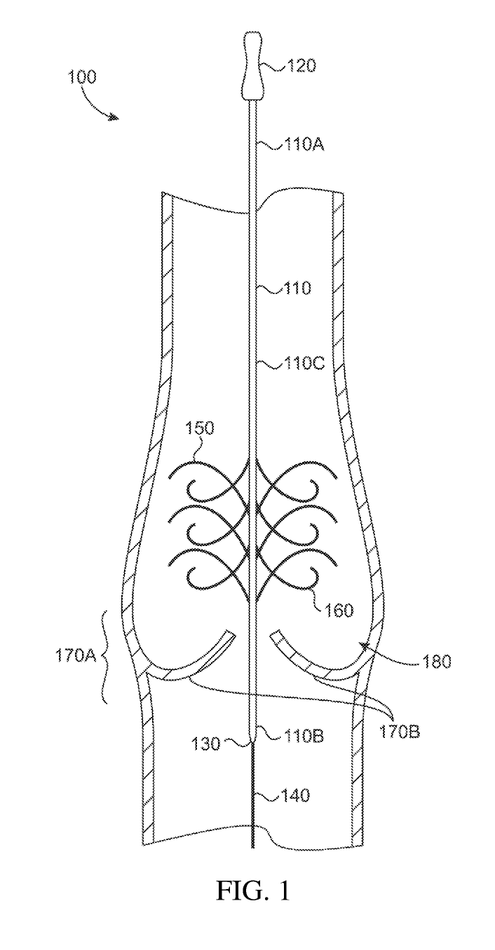

FIG. 1 depicts a preferred clot disruptor embodiment 100 with a catheter of generally hollow configuration 110, having a proximal end 110A and distal end 110B, and the shaft of which constructed from a variety of biocompatible materials and having sufficient rigidity to transmit torque during rotation of the catheter shaft. The catheter shaft has a hub 120 which remains external to the patient and is of ergonomic design to be easily gripped between thumb and forefinger. The diameter of the generally cylindrical hub is kept small to allow the maximum rotation of the shaft with a minimum of displacement with the fingers. The shaft 110C of catheter 100 has a through lumen with endhole 130 that accommodates a guidewire 140 over which the catheter may be passed. The catheter has affixed to its distal end a plurality of bristles 150 and 160, specifically designed for engagement of the vein valves 170A. In this preferred embodiment, the bristles have two general shapes, one generally curvilinear and curved back toward the hub of the catheter (160), and the other having a curvilinear configuration and angled toward the distal tip of the catheter (150). Using fluoroscopic guidance and the use of contrast to identify the location of the valves, the catheter is moved over the guidewire 140 to a point at which the bristles engage the vein valve 170A, with leaflets 170B and valve sinus 180 containing clot (not depicted). The catheter is then spun manually using hub 120 to disrupt and displace the clot at the level of the valve. The catheter may be moved linearly along the axis of the vein, back and forth, to further disrupt the clot. Once the clot is sufficiently disrupted, it may either be laced with thrombolytic agents to dissolved the now morcellated clot, or the clot may be aspirated by a variety of means through a separate device. In one preferred embodiment the inventive clot disruptor device does not include elements designed for clot removal, as such elements may increase the overall size of the catheter to an unacceptable degree; in one embodiment the inventive clot disruptor includes additional components to facilitate clot removal. When the clot has been agitated and disrupted adequately in the valve regions, the same bristles may be used for disrupting the clot in the intervening segments of vein. The bristles are designed in a way that, when they engage the valve and are pulled against it, they are sufficiently flexible that they will bend and flex at their attachment point to the catheter and pass through the valve without damaging the leaflets.

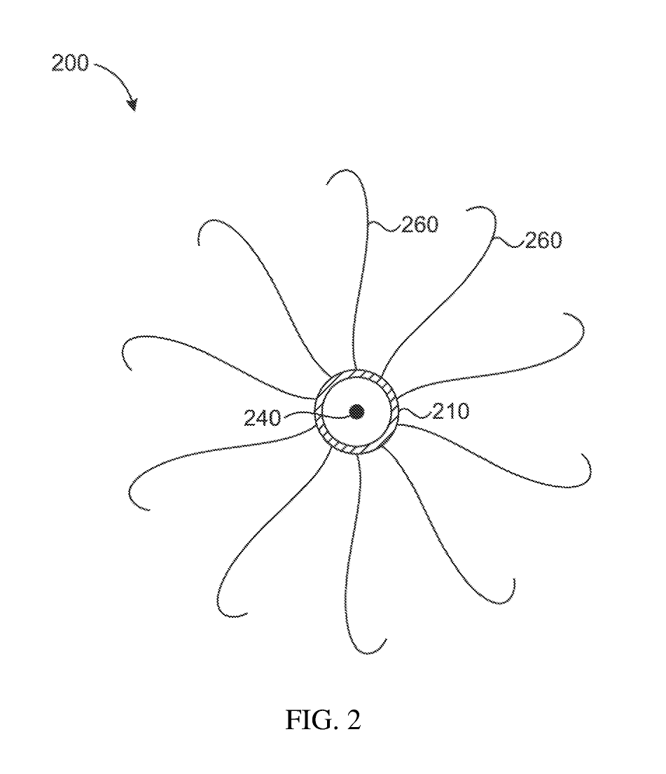

While FIG. 1 depicts bristles which are oriented in two groups diametrically opposed to one another, a preferred embodiment 200 comprises groupings of bristles (260) that are arrayed radially at lesser intervals, as depicted in FIG. 2—a cross-sectional end view of a venous clot disruptor device. The distribution of the bristles is calibrated so that there is sufficient space between and among them that the clot does not become easily trapped. Trapping a clot within the bristles may make removal of the catheter through the sheath introducer difficult.

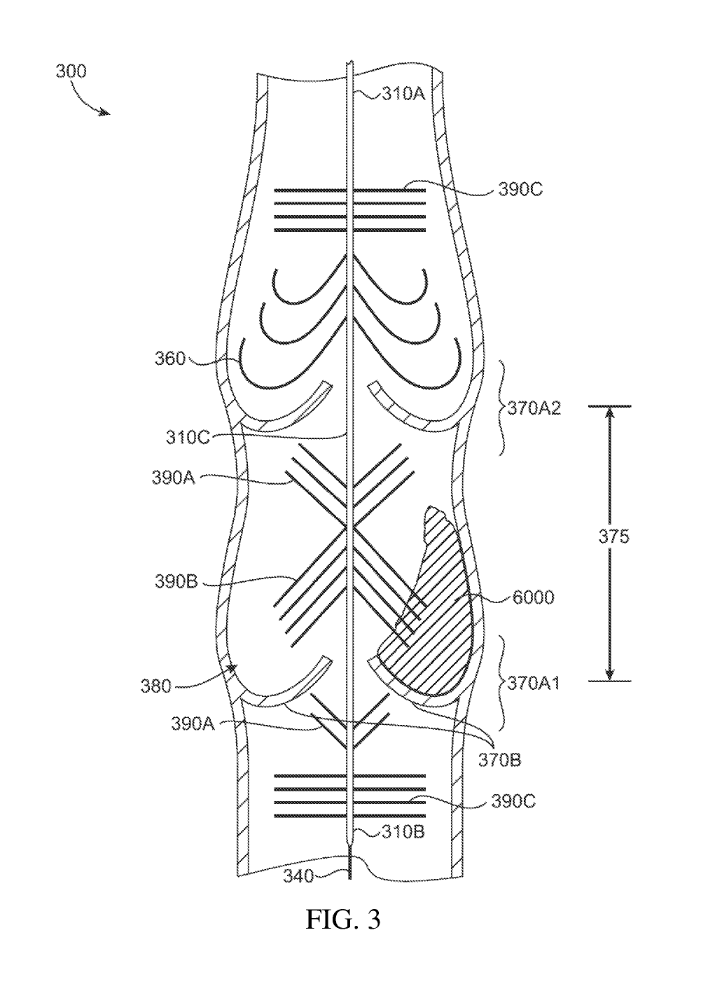

FIG. 3 depicts alternative embodiments of bristle distribution, in this case combined on a single catheter shaft 310C having a proximal end 310A and distal end 310B. These embodiments consist generally of straight bristles, some directed toward the catheter hub (390A), some directed toward the catheter tip (390B)—such that motion axially in either direction provides clot (6000) disruption on either side of a valve. Also, this configuration allows the catheter to be introduced in either direction with regard to the directionality of the valves 370A1 and 370A2. For a preferred embodiment, the catheter would consist of such bristles being limited along the shaft of the catheter over a length limited to something less than the typical spacing between venous valves (space between valve 370A1 and valve 370A2 shown as distance 375). Again, in this embodiment, the catheter shaft is moved axially in both directions by the operator, and then is spun with the bristles lodged in the valve sinuses to disrupt and displace the clot in this location. Additional straight bristles are directed perpendicular to the direction of the catheter (390C) and other bristles are generally curvilinear and curved back toward the hub of the catheter (360).

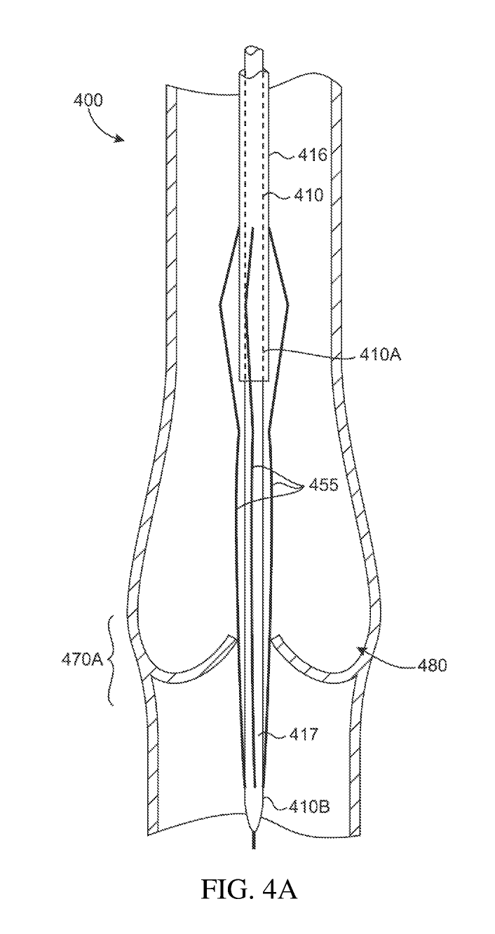

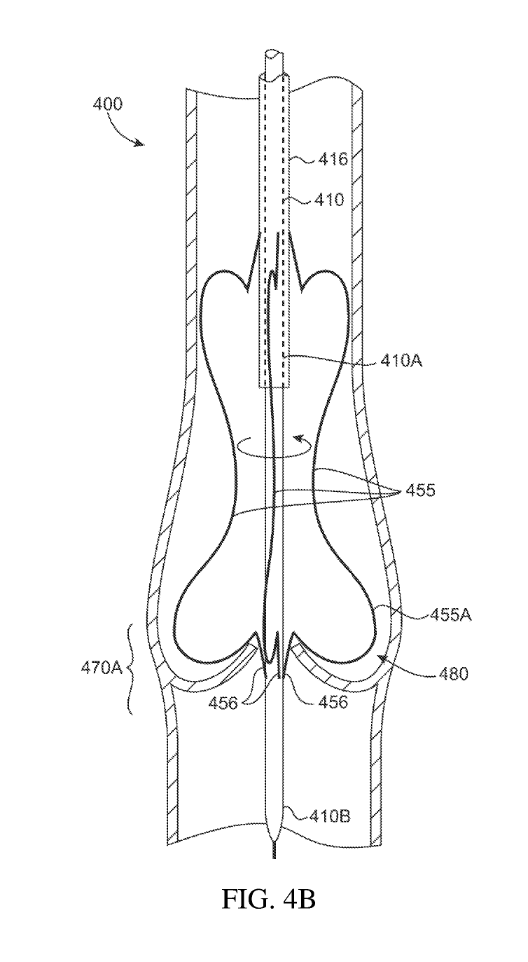

FIG. 4A and FIG. 4B depict another preferred embodiment 400 of the venous clot disruption device. This device comprises a catheter shaft 410 having two elements 416 and 417 that can slide longitudinally relative to each other. The clot disrupting filaments 455 are coupled at two points along the catheter shaft: (1) the proximal end toward the hub (410A) attached to sliding component 416 and (2) the distal end toward the catheter tip (410B) attached to sliding element 417. When this embodiment of the catheter is introduced, it is advanced as depicted in FIG. 4A, with the sliding element 416 having been withdrawn, thereby drawing the filaments 455 inward toward the catheter shaft 410. When the appropriate level within the vein is reached, the sliding element 416 is pushed inward distally, shortening the distance covered by the filaments 455 and allowing them to assume their unconstrained shape as filament blades specifically designed to engage a valve (470A). In this preferred embodiment, the filaments 455 are composed of a shape memory alloy such as nitinol.

When the device 400 is introduced and positioned properly, and the aforementioned shortening performed, the filaments 455, warmed to body temperature, take on their predetermined blade shape as depicted in FIG. 4B. The embodiment includes multiple radially arrayed filament blades 455 circumferentially about the catheter shaft. The shape of the filaments 418 is specific to the vein valve 470A, with the curved component 455A of a size and configuration that engages the valve sinus 480 as shown in FIG. 4B. Additionally, the filament ends 456 are configured to have an acute angle with regard to the catheter shaft, which allows the catheter to pass through the vein valves and passively collapses the wider element of the filaments as the catheter is advanced.

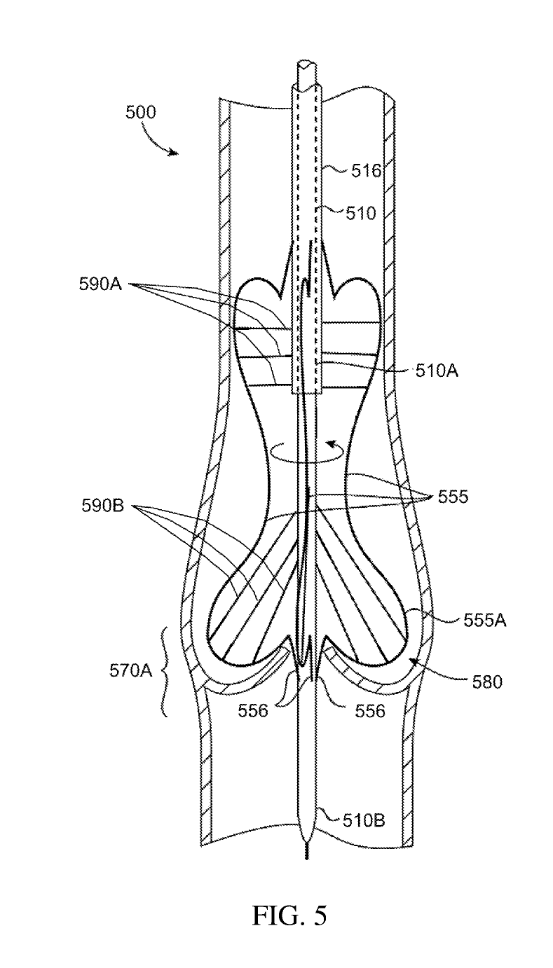

FIG. 5 depicts another preferred embodiment 500 of the venous clot disruption device, similar to embodiment 400. This device comprises a catheter shaft 510 having two elements 516 and 517 that can slide longitudinally relative to each other. The clot disrupting filaments 555 are coupled at two points along the catheter shaft: (1) the proximal end toward the hub (510A) attached to sliding component 516 and (2) the distal end toward the catheter tip (510B) attached to sliding element 517. When this embodiment of the catheter is introduced, sliding element 516 is withdrawn so that the filaments 555 are adjacent to catheter shaft 510. To deploy at the valve (570A), sliding element 516 is pushed inward distally, shortening the distance covered by the filaments 555 and allowing them to assume their unconstrained shape as filament blades with curved component 555A specifically designed to engage a valve sinus 580.

This embodiment includes multiple radially arrayed filament blades 555 positioned circumferentially about the catheter shaft. Each filament blade 555 includes additional elements 590A and 590B. Elements 590A are positioned perpendicularly to catheter shaft 510 and substantially parallel to each other; elements 590A are coupled at one end to filament blade 555 and at the opposite end to sliding component 516. Elements 590B are positioned at a non-perpendicular angle with respect to catheter shaft 510; elements 590B are coupled at one end to filament blade 555 and at the opposite end to the catheter shaft at a location proximal to where filament ends 556 are coupled. Filament ends 556 are have an acute angle with regard to the catheter shaft to facilitate catheter advancement and placement.

In view of the above, it will be seen that the several advantages of the disclosure are achieved and other advantageous results attained. As various changes could be made in the above devices and methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

While various embodiments have been described above, it should be understood that such disclosures have been presented by way of example only, and are not limiting. Thus, the breadth and scope of the subject methods, devices, and systems should not be limited by any of the above-described exemplary embodiments, but should be defined only in accordance with the following claims and their equivalents.

Having now fully described the subject methods, devices, and systems, it will be understood by those of ordinary skill in the art that the same can be performed within a wide and equivalent range of conditions, formulations and other parameters without affecting their scope or any embodiment thereof. All cited patents, patent applications and publications are fully incorporated by reference in their entirety.

1. A medical device comprising:
A catheter having a longitudinal axis with a distal end and a proximal end;
the catheter comprising a catheter tip at the distal end of the catheter and comprising a circumferential cylindrical hub at the distal end of the catheter proximal to the catheter tip, the hub movable along the longitudinal axis between a first position proximal to the catheter tip and a second position distal to the catheter tip;
flexible filaments having a distal end and a proximal end, where the proximal end of at least one of the filaments is connected to the hub;
where said distal end comprises filaments positioned about the circumference of the catheter that engages and disrupts a venous clot.

2. A medical device of claim 1 where said bristles the distal end of the filaments are coupled to the catheter.

3. A medical device of claim 1 where said filaments comprise curvilinear bristles.

4. A medical device of claim 1 where said filaments comprise angled filaments.

5. A medical device of claim 1 where said filaments comprise curvilinear filaments and straight filaments.

6. A medical device of claim 1 where said filaments comprise curvilinear filaments curved toward the proximal end of the catheter.

7. A medical device of claim 1 where said filaments comprise curvilinear filaments shaped to engage a venous valve.

8. A medical device of claim 1 comprising a cluster of filaments positioned radially about a circumference of the catheter.

9. (canceled)

10. (canceled)

11. A medical device of claim 1 where said filaments are comprised of a material selected from the group consisting of metallic material ; shape memory alloy, nickel and titanium alloy, nylon and polymeric material.

12. (canceled)

13. (canceled)

14. A medical device of claim 1 where said filaments are looped.

15. A medical device comprising:
a catheter having a longitudinal axis with a distal end and a proximal end,
said catheter comprising a catheter tip at the distal end of the catheter and comprising a hub at the distal end of the catheter proximal to the catheter tip, said hub movable along the longitudinal axis between a first position proximal to the catheter tip and a second position distal to the catheter tip;
and one or more than one filament having a distal end and a proximal end, where the proximal end of the one or more than one filament is coupled to the hub and the distal end of the one or more than one filament is coupled to the tip of the catheter; and
where said one or more than one filament comprises a flexible material.

16. A medical device of claim 15 comprising between one and thirty filaments.

17. (canceled)

18. (canceled)

19. (canceled)

20. A medical device of claim 15 where said one or more than one filament comprises a material selected from the group consisting of a polymeric material, a metallic material, or a shape memory alloy.

21. (canceled)

22. (canceled)

23. (canceled)

24. A medical device of claim 15 where said one or more then one filament expands radially outward from the longitudinal axis of the catheter when the hub is in a second position.

25. (canceled)

26. A medical device of claim 15 where said one or more than one filament further comprises a web of flexible filament elements.

27-44. (canceled)

45. A medical device comprising:
a catheter having a longitudinal axis with a distal end and a proximal end;
the catheter comprising a catheter tip at the distal end of the catheter and comprising a hub at the distal end of the catheter proximal to the catheter tip, said hub movable along the longitudinal axis between a first position proximal to the catheter tip and a second position distal to the catheter tip;
and one or more than one filament having a distal end and a proximal end, where the proximal end of the one or more than one filament is coupled to the hub and the distal end of the one or more than one filament extends toward the tip of the catheter; and
where said one or more than one filament comprises a flexible material.

46. A medical device of claim 45 where said one or more than one filament comprises a material selected from the group consisting of a polymeric material, a metallic material, nylon, polymeric material or a shape memory alloy.

47. A medical device of claim 45 wherein the filaments have a shape memory of J-shaped curve.

48. A medical device of claim 45 further comprising a second set of filaments orthogonally disposed to the catheter proximal to the hub.

49. A medical device of claim 45 further comprising a second set of filaments orthogonally disposed to the catheter distal to the hub.
